(19) United States
(12) Patent Application Publication
(10) Pub. No.: US 2012/0122993 A1
(43) Pub. Date: May 17, 2012

(54) PHARMACEUTICAL COMPOSITION CONTAINING 1H-INDEN-1-AMINE, 2,3-DIHYDRO-N-2-PROPYNYL-(1R)-, METHANESULFONATE

(75) Inventors: Kamalakar Talasila, Thane (IN); Hemant Narkhede, Thane (IN); Atul Daroi, Thane (IN)

(73) Assignee: SANDOZ AG, Basel (CH)

(21) Appl. No.: 13/202,583

(22) PCT Filed: Mar. 4, 2010
(86) PCT No.: PCT/EP2010/052744

§ 371 Date: Nov. 28, 2011

(30) Foreign Application Priority Data
Mar. 5, 2009 (IN) 478/MUM/2009

(51) Int. Cl. A61K 31/13; A61P 25/00; A61P 25/24; A61P 25/16; A61P 25/28

(52) U.S. Cl. 514/661

(57) ABSTRACT

A pharmaceutical composition comprising as an active ingredient a therapeutically effective amount of R(+)-N-propargyl-1-aminoindan mesylate thereof, and less than 50% by weight of hexahydric sugar alcohols.

### FIELD OF THE INVENTION

The present invention relates to pharmaceutical compositions containing R(+)N-propargyl-1-aminoindan mesylate, which is particularly useful for the treatment of Parkinson's disease, memory disorders and dementia of the Alzheimer type (DAT), depression, and hyperactive syndrome in children.

### BACKGROUND OF THE INVENTION

Rasagiline Mesylate (1H-Inden-1-Amine, 2,3-Dihydro-N-2-Propynyl-, (1R)-, Methanesulfonate) has the following structure,

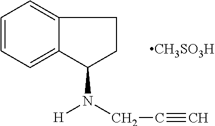

Rasagiline is known in literature as an irreversible inhibitor of monoamine oxidase used as a monotherapy in early Parkinson's disease or as an adjunct therapy in more advanced cases. It is selective for monoamine oxidase type B.

U.S. Pat. No. 5,532,415 discloses R(+)-N-propargyl-1-aminoindan, its preparation, and various pharmaceutically acceptable salts thereof.

U.S. Pat. No. 6,126,968 (the '968 patent) discloses pharmaceutical compositions comprising R(+)PAI. R(+)PAI and salts thereof have been shown to be selective inhibitors of MAO-B, useful in treating Parkinson's disease and various other conditions. The ('968) Patent also claims a pharmaceutical composition comprising as an active ingredient a therapeutically effective amount of a R(+)-N-propargyl-1-aminoindan or a pharmaceutically acceptable salt thereof, and at least 60% by weight of at least one alcohol being a member selected from the group of pentahydric and hexahydric alcohols. According to this patent, where the amount of said at least one alcohol is less than 70% by weight, the composition further comprises citric acid in an amount of 0.5 to 2% by weight of the total composition. Where the amount of said at least one alcohol is at least 70% by weight, the inclusion of citric acid is optional.

United States Publication number 2006/0188581 provides a pharmaceutical preparation of R(+)-N-propargyl-1-aminoindan salts having enhanced content uniformity, which comprises reducing the particle size of a pharmaceutically acceptable salt of R(+)-N-propargyl-1-aminoindan to a particle size of less than 250 microns. The present invention is to provide content uniformity of drug products comprising R(+)-N-propargyl-1-aminoindan, comprising milling R(+) particles to reduce particle size.

Chinese Publication Number 11152153 discloses an oral solid medicinal combination, comprises 0.5 weight percent to 3 weight percent of rasagiline and salt of rasagiline, be not less than 40 percent and less than 60 weight percent of pentahydric alcohol and/or hexahydric alcohol and 0.5 weight percent to 3 weight percent of organic acid.

Further the pharmaceutical compositions known in the prior art lack stability and produce impurities in excess of the pharmaceutical limit over a certain period of time making it unfit for consumption.

In light of the above disadvantage, there remains a need for formulations which are stable over a longer period of time.

The present inventors have surprisingly developed a stable formulation comprising of rasagiline mesylate and less than 50% of sugar alcohol. The present inventors have surprisingly found that the formulation developed in the present invention is stable and the amount of impurity developed over a period of six months is very less.

### OBJECT OF THE INVENTION

The object of the present invention is to provide stable formulations comprising an effective amount of R(+)-N-propargyl-1-aminoindan mesylate.

### SUMMARY OF THE INVENTION

According to an aspect of the invention, there is provided a pharmaceutical composition comprising as an active ingredient a therapeutically effective amount of R(+)-N-propargyl-1-aminoindan salt thereof, and less than 50% by weight of hexahydric sugar alcohols.

### DETAILED DESCRIPTION OF THE INVENTION

The present invention provides a pharmaceutical composition comprising as an active ingredient a therapeutically effective amount of a compound R(+)-N-propargyl-1-aminoindan mesylate thereof, and less than 50% by weight of hexahydric sugar alcohols. The present invention provides an oral pharmaceutical dosage form comprising R(+)-N-propargyl-1-aminoindan mesylate.

In one embodiment, the oral pharmaceutical dosage form is a tablet.

In yet another embodiment, the hexahydric sugar alcohol is selected from a group consisting of mannitol, xylitol, sorbitol, maltitol, isomalt, lactitol monohydrate.

In another embodiment, the hexahydric sugar alcohol is selected from a group consisting of Mannitol, Sorbitol, Xylitol.

In yet another embodiment, the preferred hexahydric sugar alcohol is mannitol and the amount of said hexahydric sugar alcohol is less than 50% by weight of the total composition.

In yet another embodiment, the particle size of R(+)-N-propargyl-1-aminoindan in the formulation is less than 200 microns that is d90 is NMT 200 microns.

The composition of the present invention may also include pharmaceutically acceptable excipients such as fillers, lubricants, disintegrants, binders, diluents and glidants.

Binders which could be used include, but are not limited to, Starches, e.g., Potato Starch, Wheat Starch, Corn Starch, Pre-gelatinized Starch; Gums, such as Gum Tragacanth, Acacia Gum and Gelatin; and Polyvinyl Pyrrolidone, Cellulose Polymers like Methyl Cellulose, Ethyl Cellulose, Propyl Cellulose, Hydroxymethyl Cellulose, Hydroxyethyl Cellulose, Hydroxypropyl Cellulose, Hydroxypropyl Methyl Cellulose, Polyvinyl Alcohol etc

Fillers which could be used include, but are not limited to, Microcrystalline Cellulose [Avicel PH-101, Avicel PH-301, Avicel PH-102 Scg, Avicel HFE-102, Avicel PH-200 Avicel PH-302], Starch, Pre-Gelatinized Starch, Modified Starch, Dibasic Calcium Phosphate Dihydrate, Calcium Sulfate Trihydrate, Calcium Sulfate Dihydrate, Calcium Carbonate, Calcium Phosphate Anhydrous, Dextrose, Sucrose, Lactose, Mannitol And Sorbitol, Xylitol, Maltitol, Isomalt, Erythritol, Lactitol Monohydrate, Dextrin, Malto dextrin and, Cyclodextrins. Coprocessed materials Eg like Starlac, Prosolv, Avicel CE15, Ludipress, F-Melt type C & D, Dipac, EMDEX®, SUGARTAB®, Ditab, Pharmatose DCL 40, Pharmaburst, Starlac, Advantose, Panexcea MHC 333G, Xylitab®, Ludiflash, and Avicel HFE.

Preferred diluents include, but are not limited to, Dextrose, Sorbitol, Sucrose, Lactose, Lactose Monohydrate, Mannitol, Gelatin, Starch, Dextrin, Malto Dextrin, Cyclodextrins, Microcrystalline cellulose, Pre-gelatinized starch, Modified starch, Dibasic Calcium Phosphate dihydrate, Calcium Sulfate Trihydrate, Calcium Sulfate Dihydrate, Calcium Carbonate, Calcium Phosphate anhydrous, Xylitol, Maltitol, Isomalt, Erythritol, Lactitol Monohydrate, Dextrin, Malto dextrin and Cyclodextrins. Coprocessed materials Eg like Starlac, Prosolv, Avicel CE15, Ludipress, F-Melt type C & D, Dipac, EMDEX®, SUGARTAB®, Ditab, Pharmatose DCL 40, Pharmaburst, Starlac, Advantose, Panexcea MHC 333G, Xylitab®, Ludiflash, and Avicel HFE.

Disintegrants which could be used include but are not limited to natural Starches, such as Maize Starch, Potato Starch and the like, directly compressible Starches, e.g., Sta-rx® 1500; Modified Starches, e.g., Carboxymethyl Starches and Sodium Starch Glycolate, available as Primojel®, Explotab®, Explosol®; and starch derivatives, such as Amylase. Cross-linked polyvinylpyrrolidones, e.g., crospovidones, such as Polyplasdone® XL and Kollidon® CL. Alginic acid and sodium alginate. Methacrylic acid-divinylbenzene co-polymer salts, Cross-linked sodium carboxymethylcellulose, available as, e.g., Ac-di-sol®, Primellose®, Pharmacel® XL, Explocel® and Nymcel® ZSX. Additional disintegrants also include Hydroxypropyl Cellulose, Hydroxypropylmethyl Cellulose, Croscarmellose Sodium, Sodium Starch Glycolate, Polacrillin, Potassium Polyacrylates, such as Carbopol®, Magnesium Aluminium Silicate and Bentonite.

Lubricants include but are not limited to stearate salts of metals e.g. Magnesium Stearate, Sodium Stearyl Fumarate, Hydrogenated Vegetable oil Type I and II (eg: Lubritab, Sterotex grades, Castorwax), Glyceryl dibehanate, Calcium stearate, zinc stearate, Stearic acid.

Glidents include but are not limited to colloidal silicon dioxide, Magnesium Trisilicate, Magnesium Silicate, Cellulose, Talc and Starch.

The following non-limiting examples are given by way of illustration.

### Example 1

| Sr. No | Ingredients | Mg/0.5 mg tab | Mg/1 mg tab | % w/w |
|---|---|---|---|---|
| 1 | Rasagiline mesylate | 0.78 | 1.56 | 1.30 |
| 2 | Mannitol | 29.12 | 58.24 | 48.53 |
| 3 | Maize starch | 22.6 | 45.20 | 37.67 |
| 4 | Pre gelatinized starch | 6.00 | 12.00 | 10.00 |
| 5 | Colloidal silicon | 0.30 | 0.60 | 0.50 |
| | dioxide | | | |
| 6 | Talc | 0.60 | 1.20 | 1.00 |
| 7 | Stearic acid | 0.60 | 1.20 | 1.00 |
| | Tablet weight | 60.00 | 120.00 | 100.00 |

Manufacturing process:
1. Mannitol, corn starch and pregelatinized starch were sifted and mixed in a suitable mixer,
2. Rasagiline was dissolved in purified water,
3. Mannitol, corn starch, pregelatinized starch were granulated using Rasagiline solution and the granules were dried in suitable drier,
4. The dried granules were screened,
5. Colloidal silicon dioxide, talc and Stearic acid were sifted;
6. The screened granules were mixed with colloidal silicon dioxide and was lubricated with talc and Stearic acid;
7. The lubricated mixture was compressed into tablet.

### Example 2

| S. No. | Ingredients | Mg/0.5 mg tab | Mg/1 mg tab | % w/w |
|---|---|---|---|---|
| 1 | Rasagiline mesylate | 0.78 | 1.56 | 1.30 |
| 2 | Mannitol | 29.12 | 58.24 | 48.53 |
| 3 | Corn starch | 22.00 | 44.00 | 36.67 |
| 4 | Pregelatinised starch | 6.00 | 12.00 | 10.00 |
| 5 | Colloidal silicon | 0.30 | 0.60 | 0.50 |
| | dioxide | | | |
| 6 | Talc | 0.90 | 1.80 | 1.50 |
| 7 | Stearic acid | 0.90 | 1.80 | 1.50 |
| | Tablet weight | 60.00 | 120.00 | 100.00 |

Manufacturing process:
1. Rasagiline, Mannitol, corn starch and pregelatinized starch and part of Colloidal silicon dioxide were sifted and mixed in a suitable mixer,
2. Dry mix was granulated using purified water and the granules were dried in suitable drier,
3. The dried granules were screened,
4. Remaining part of Colloidal silicon dioxide, talc and Stearic acid were sifted;
5. The screened granules were mixed with colloidal silicon dioxide and was lubricated with talc and Stearic acid;
6. The lubricated mixture was compressed into tablet.

The stability data for different batches of Rasagiline as checked from time to time is given below in Table 1

TABLE 1
Stability data for the formulation of present invention

| | | | | | Related Substances | | | |
|---|---|---|---|---|---|---|---|---|
| B. No. | Strength | Condition | Pack | RRT | 0.27 | 0.49 | 0.77 | Total |
| KT-09- | 1 mg | Initial | | % Impurity | ND | ND | ND | 0.0 |
| 107 | | 40° C., 75% | HDPE | | 0.09 | 0.03 | ND | 0.12 |
| | | RH-3 M | | | | | | |
| | | 25° C., 60% | HDPE | | ND | ND | ND | 0.0 |
| | | RH-3 M | | | | | | |
| | | 40° C., 75% | HDPE | | 0.23 | 0.05 | ND | 0.28 |
| | | RH-6 M | | | | | | |
| | | 25° C., 60% | HDPE | | BLOQ | ND | ND | BLOQ |
| | | RH-6 M | | | | | | |
| 385/084 | 1 mg | Initial | | | BLOQ | ND | ND | BLOQ |
| | | 40° C., 75% | HDPE | | 0.10 | BLOQ | ND | 0.10 |
| | | RH-3 M | | | | | | |
| | | 25° C., 60% | HDPE | | ND | ND | ND | 0.0 |
| | | RH-3 M | | | | | | |
| | | 40° C., 75% | HDPE | | 0.17 | 0.06 | ND | 0.23 |
| | | RH-6 M | | | | | | |
| | | 25° C., 60% | HDPE | | BLOQ | ND | ND | BLOQ |
| | | RH-6 M | | | | | | |
| 385/089 | 0.5 mg | Initial | | | BLOQ | ND | ND | BLOQ |
| | | 40° C., 75% | HDPE | | 0.08 | BLOQ | ND | 0.08 |
| | | RH-3 M | | | | | | |
| | | 25° C., 60% | HDPE | | ND | ND | ND | 0.0 |
| | | RH-3 M | | | | | | |
| | | 40° C., 75% | HDPE | | 0.16 | 0.05 | ND | 0.22 |
| | | RH-6 M | | | | | | |
| | | 25° C., 60% | HDPE | | BLOQ | ND | ND | BLOQ |
| | | RH-6 M | | | | | | |
| 385/091 | 0.5 mg | Initial | | | BLOQ | ND | ND | BLOQ |
| | | 40° C., 75% | HDPE | | 0.10 | BLOQ | ND | 0.10 |
| | | RH-3 M | | | | | | |
| | | 25° C., 60% | HDPE | | ND | ND | ND | 0.0 |
| | | RH-3 M | | | | | | |
| | | 40° C., 75% | HDPE | | 0.17 | 0.06 | ND | 0.23 |
| | | RH-6 M | | | | | | |
| | | 25° C., 60% | HDPE | | BLOQ | ND | ND | BLOQ |
| | | RH-6 M | | | | | | |

ND: Not detected
BLOQ refers to Below Limit of Quantification
HDPE refers to High Density Polyethylene

A stability study for the formulations provided in U.S. Pat No. 6,126,968 is given in table 2.

TABLE 2
Formulation as given in U.S. Pat. No. 6,126,968

| B. No. | Strength | Condition | Pack | Related Substances | | | | | Total |
|---|---|---|---|---|---|---|---|---|---|
| Example 2 | 1 mg | | | RRT | 1.17 | 1.81 | 3.52 | 5.24 | |
| | | 40° C., 75% | HDPE | % Impurity | 0.22 | 1.19 | 0.22 | 0.4 | 2.1 |
| | | RH-2 M | | | | | | | |
| Example 3 | 1 mg | | | RRT | 1.17 | 1.81 | 3.53 | 5.22 | |
| | | 40° C., 75% | HDPE | % Impurity | 0.04 | 0.38 | 0.04 | 0.12 | 0.72 |
| | | RH-2 M | | | | | | | |
| Example 5 | 1 mg | | | RRT | 0.95 | 1.17 | 1.83 | 3.55 | |
| | | 40° C., 75% | HDPE | % Impurity | 0.06 | 0.07 | 0.49 | 0.08 | 0.75 |
| | | RH-2 M | | | | | | | |

From table 1 and table 2, it can be observed that the amount of impurities formed in the Sandoz formulation is less than that obtained in U.S. 6,126,968. It can be concluded that the Sandoz formulation has better stability as compared to U.S. 6,126,968 formulation.

The dissolution studies with regards to the Sandoz formulation is given in table 3.

TABLE 3
Rasagiline Tablet Dissolution Result
Dissolution Condition: 500 ml 0.1N HCl, USP II Apparatus, 50 RPM

% Drug
Released

| Strength | B. No. | 15 min | 30 min |
|---|---|---|---|
| 1 mg | 385/084 | 97 | 99 |
| 1 mg | KT-09-107 | 94 | 97 |
| 0.5 mg | 385/089 | 104 | 107 |
| 0.5 mg | 385/091 | 93 | 102 |

1-6. (canceled)

7. A pharmaceutical composition comprising as an active ingredient a therapeutically effective amount of R(+)-N-propargyl-1-aminoindan mesylate (Rasagiline mesylate), and less than 50% by weight of hexahydric sugar alcohols, wherein the amount of Rasagiline mesylate present in the total composition is 1.3% by weight.

8. The pharmaceutical composition according to claim 7, wherein the hexahydric sugar alcohol is selected from the group consisting of mannitol, and sorbitol.

9. The pharmaceutical composition according to claim 8, wherein the hexahydric sugar alcohol is mannitol.

10. The pharmaceutical composition according to claim 9, further comprising a pharmaceutically acceptable excipient.

11. The pharmaceutical composition according to claim 10, wherein the pharmaceutically acceptable excipient is selected from the group consisting of fillers, lubricants, disintegrants, binders, diluents, and glidants.

12. The pharmaceutical composition according to claim 10, in the form of a tablet.
